(19) United States
(12) Patent Application Publication
(10) Pub. No.: US 2020/0411145 A1
(43) Pub. Date: Dec. 31, 2020

(54) MEDICAL CARE SUPPORT DEVICE, OPERATION METHOD OF MEDICAL CARE SUPPORT DEVICE, AND OPERATION PROGRAM OF MEDICAL CARE SUPPORT DEVICE

(71) Applicant: FUJIFILM Corporation, Tokyo (JP)

(72) Inventors: Akemi ODA, Kanagawa (JP); Hirona YUMBE, Kanagawa (JP); Eiichi IMAMICHI, Kanagawa (JP); Shigetoshi ISHIKAWA, Kanagawa (JP); Takeshi FUKUDA, Kanagawa (JP); Haruyasu NAKATSUGAWA, Kanagawa (JP)

(73) Assignee: FUJIFILM Corporation, Tokyo (JP)

(21) Appl. No.: 16/900,284

(22) Filed: Jun. 12, 2020

(30) Foreign Application Priority Data
Jun. 26, 2019 (JP) 2019-118511

(51) Int. Cl. G16H 10/60; G16H 15/00; G16H 70/20; H04L 29/06; A61B 6/00

(52) U.S. Cl. CPC G16H 10/60; G16H 15/00; G16H 70/20; A61B 6/465; H04L 63/102; A61B 6/508; H04L 63/083

(57) ABSTRACT

A CPU of a medical care support server functions as a reception unit, an RW control unit, and a screen output control unit. The reception unit receives precaution information on an owner of a pet as owner information. The RW control unit performs control to store the precaution information in a storage device. The screen output control unit outputs a pet health management screen on which a balloon display region, which is a display region dedicated to the precaution information, is provided. An information mark indicating presence of the precaution information is displayed on the health management screen. The balloon display region is displayed only in a case where the information mark is selected.

### CROSS-REFERENCE TO RELATED APPLICATIONS

The present application claims priority under 35 U.S.C. § 119 to Japanese Patent Application No., 2019-118511 filed on Jun. 26, 2019. The above application is hereby expressly incorporated by reference, in its entirety, into the present application.

### BACKGROUND

### 1. Technical Field

The technique of the present disclosure relates to a medical care support device, an operation method of a medical care support device, and an operation program of a medical care support device.

### 2. Description of the Related Art

In an animal hospital, various types of medical care support using a computer is performed. For example, JP2012-146029A describes a medical care support device that has a function of allowing a veterinarian to input comments such as operation contents and findings, and displays the comments together with pet medical record information.

### SUMMARY

A pet medical treatment cost may be unexpectedly high since the treatment cost is not covered by insurance. Therefore, trouble over the medical treatment cost is often caused by a pet owner. Therefore, there has been a known request from veterinarians to want to know precaution information on the pet owner, such as a need to explain the medical treatment cost.

With the technique described in JP2012-146029A, it is possible to display the precaution information on the pet owner by using a comment function. However, the precaution information may be buried in other comments such as operation contents and findings, and thus the veterinarian may overlook the precaution information.

An object of the technique of the present disclosure is to provide a medical care support device, an operation method of a medical care support device, and an operation program of a medical care support device, which can prevent precaution information on a pet owner from being overlooked.

In order to achieve the above object, a medical care support device according to the present disclosure comprises a reception unit that receives precaution information on an owner of a pet as owner information, a storage control unit that performs control to store the precaution information, and a screen output control unit that performs control to output a health management screen of the pet, on which a display region dedicated to the precaution information is provided.

It is preferable that the screen output control unit displays the display region only in a case where there is an instruction to display the precaution information.

It is preferable that the screen output control unit displays a mark indicating presence of the precaution information on the health management screen in a state where the display region is not displayed.

It is preferable that the precaution information is input by being selected from a plurality of options prepared in advance.

It is preferable that the reception unit receives pet information which is information relating to the pet, the storage control unit performs control to store the owner information and the pet information in association with each other, and the screen output control unit displays the owner information and the pet information on the health management screen.

It is preferable that the pet information includes medical record information of the pet.

It is preferable that the pet information includes character information of the pet.

It is preferable that the screen output control unit displays the character information on the health management screen only in a case where there is an instruction to display the character information.

It is preferable that the screen output control unit displays a transition in the character information on the health management screen.

It is preferable that the pet information includes breeding environment information of the pet.

It is preferable that the pet information includes stress information of the pet.

It is preferable that there is a browsing restriction on the health management screen by authentication, and the screen output control unit outputs the health management screen only in a case where the authentication is permitted.

An operation method of a medical care support device according to the present disclosure comprises a reception step of receiving precaution information on an owner of a pet as owner information, a storage control step of performing control to store the precaution information, and a screen output control step of performing control to output a health management screen of the pet, on which a display region dedicated to the precaution information is provided.

An operation program of a medical care support device according to the present disclosure that causes a computer to function as a reception unit that receives precaution information on an owner of a pet as owner information, a storage control unit that performs control to store the precaution information, and a screen output control unit that performs control to output a health management screen of the pet, on which a display region dedicated to the precaution information is provided.

With the technique of the present disclosure, it is possible to provide the medical care support device, the operation method of the medical care support device, and the operation program of the medical care support device, which can prevent the precaution information on the pet owner from being overlooked.

### BRIEF DESCRIPTION OF THE DRAWINGS

Exemplary embodiments according to the technique of the present disclosure will be described in detail based on the following figures, wherein:

FIG. 1 is a diagram showing a medical care support system;

FIG. 2 is a diagram showing medical care information;

FIG. 3 is a diagram showing owner basic information;

FIG. 4 is a diagram showing precaution information;

FIG. 5 is a diagram showing pet basic information;

FIG. 6 is a diagram showing medical record information;

FIG. 7 is a diagram showing character information;

FIG. 8 is a diagram showing breeding environment information;

FIG. 9 is a diagram showing pet food information;

FIG. 10 is a diagram showing medication information;

FIG. 11 is a diagram showing association between owner information and pet information;

FIG. 12 is a block diagram showing a computer constituting a medical care support server and a doctor terminal;

FIG. 13 is a block diagram showing a processing unit of a CPU of the medical care support server;

FIG. 14 is a block diagram showing a processing unit of a CPU of the doctor terminal;

FIG. 15 is a diagram showing an authentication screen;

FIG. 16 is a diagram showing a precaution information input screen;

FIG. 17 is a diagram showing a designation screen;

FIG. 18 is a diagram showing an outline of a health management screen;

FIG. 19 is a diagram showing a first display region of the health management screen;

(a) and (b) of FIG. 20 are diagrams showing a second display region of the health management screen. (a) of FIG. 20 shows a state where a balloon display region, which is a display region dedicated to the precaution information, is not displayed, and (b) of FIG. 20 shows a state where the balloon display region is displayed, respectively;

FIG. 21 is a diagram showing a third display region of the health management screen;

FIG. 22 is a diagram showing a fourth display region of the health management screen;

FIG. 23 is a diagram showing a fifth display region of the health management screen;

FIG. 24 is a diagram showing a sixth display region of the health management screen;

FIG. 25 is a diagram showing a seventh display region of the health management screen;

FIG. 26 is a diagram showing an eighth display region of the health management screen;

FIG. 27 is a diagram showing a ninth display region of the health management screen;

FIG. 28 is a flowchart showing a processing procedure of the medical care support server;

FIG. 29 is a flowchart showing a processing procedure of the medical care support server;

FIG. 30 is a flowchart showing a processing procedure of the medical care support server;

FIG. 31 is a diagram showing an example of displaying the precaution information on a pop-up screen;

FIG. 32 is a diagram showing an example of displaying a comment of the character information in the balloon display region;

(a) and (b) of FIG. 33 are diagrams showing an example of displaying the comment of the character information in the balloon display region. (a) of FIG. 33 shows a state where the balloon display region is not displayed, and (b) of FIG. 33 shows a state where the balloon display region is displayed, respectively;

FIG. 34 is a table showing the character information according to a breed;

FIG. 35 is a table showing the character information according to age;

FIG. 36 is a diagram showing a character information setting screen;

(a) and (b) of FIG. 37 are diagrams showing a second embodiment in which the character information is switched between display and non-display. (a) of FIG. 37 shows a state where a mark indicating a character level is not displayed, and (b) of FIG. 37 shows where the mark is displayed;

FIG. 38 is a diagram showing a third embodiment in which a transition in the character information is displayed;

FIG. 39 is a diagram showing an example in which a cause of the transition in the character information determined and input by a veterinarian is displayed in the balloon display region;

(a) and (b) of FIG. 40 are diagrams showing an example in which the cause of the transition in the character information determined and input by the veterinarian is displayed in the balloon display region. (a) of FIG. 40 shows a state where the balloon display region is not displayed, and (b) of FIG. 40 shows a state where the balloon display region is displayed, respectively;

FIG. 41 is a diagram showing a fourth embodiment in which pet stress information is added to pet information; and

(a), (b), and (c) of FIG. 42 are diagrams showing a display mode of the health management screen in a case where a doctor terminal is a smartphone.

### DETAILED DESCRIPTION

### First Embodiment

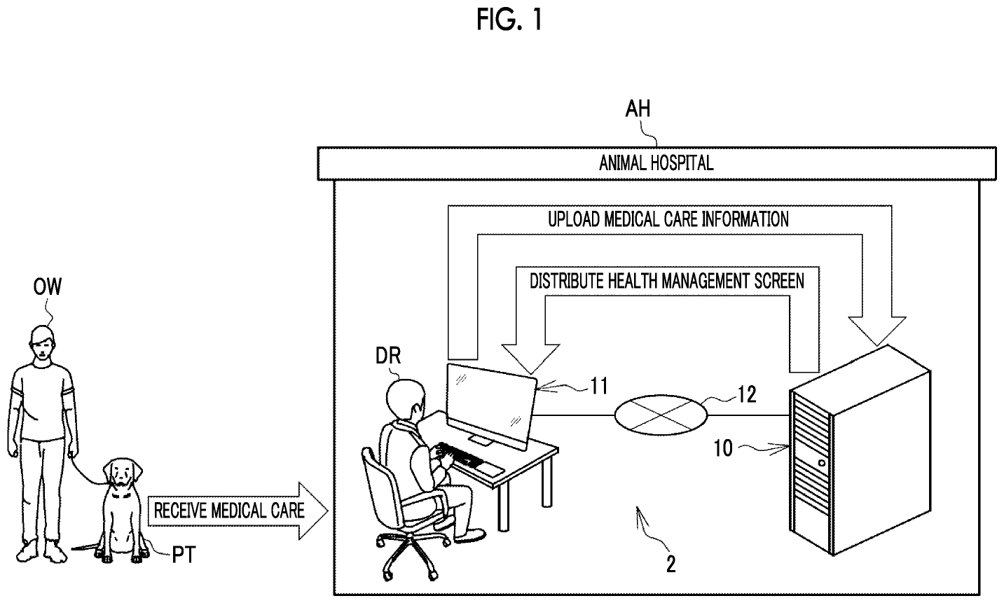

In FIG. 1, a medical care support system 2 is a system that is installed in an animal hospital AH and supports medical care of a pet PT (a dog is illustrated in FIG. 1). The medical care support system 2 comprises a medical care support server 10 and a doctor terminal 11 operated by a veterinarian DR in the animal hospital AH. The medical care support server 10 is, for example, a server computer and is an example of a “medical care support device” according to the technique of the present disclosure. The doctor terminal 11 is, for example, a desktop personal computer.

The medical care support server 10 and the doctor terminal 11 are communicably connected to each other through a network 12. The network 12 is, for example, a local area network (LAN).

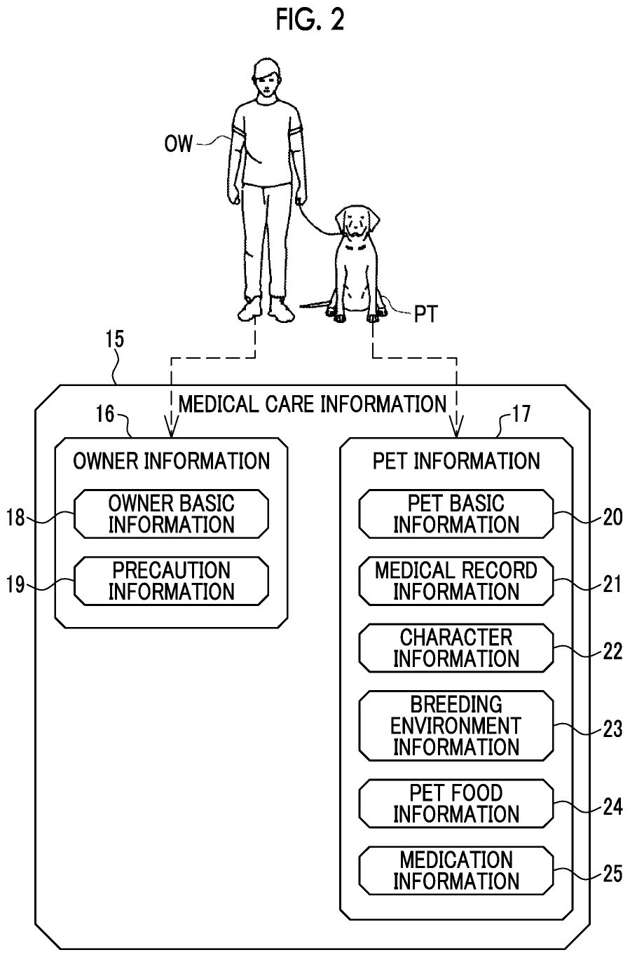

An owner OW of the pet PT comes to the animal hospital AH with the pet PT and causes the pet PT to receive the medical care of the veterinarian DR at the animal hospital AH. The veterinarian DR operates the doctor terminal 11 to input medical care information 15 (refer to FIG. 2), which is information relating to the medical care of the pet PT, and uploads the medical care information 15 to the medical care support server 10.

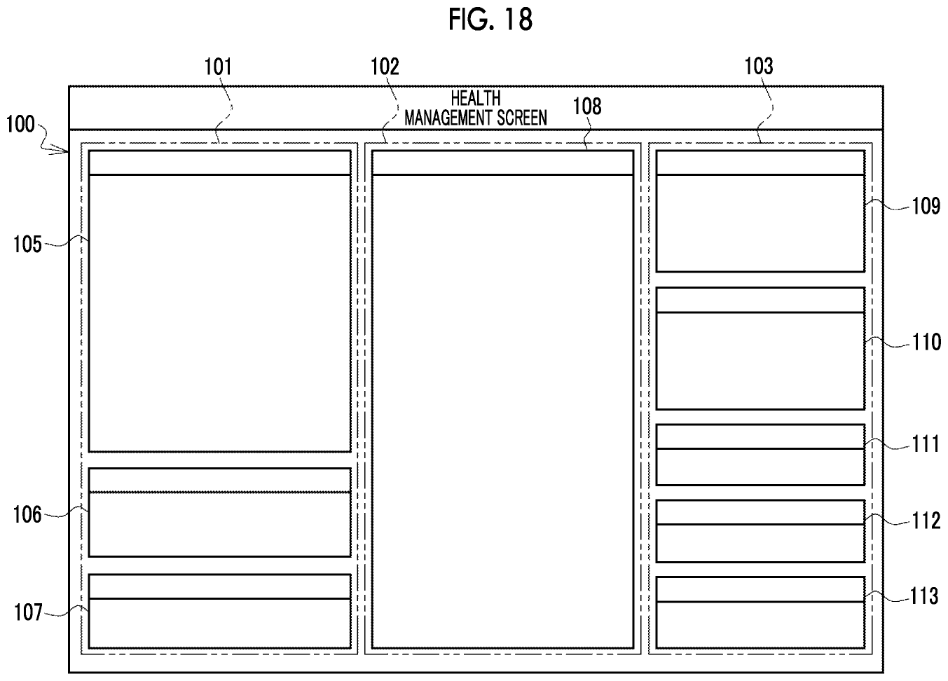

The medical care support server 10 stores the medical care information 15 uploaded from the doctor terminal 11. At the next medical care of the pet PT or the like, the veterinarian DR operates the doctor terminal 11 to transmit a request for distributing a health management screen 100 (refer to FIG. 18) of the pet PT to the medical care support server 10. The medical care support server 10 receives the distribution request, generates the health management screen 100 based on the medical care information 15, and distributes the generated health management screen 100 to the doctor terminal 11. The doctor terminal 11 displays the health management screen 100 distributed from the medical care support server 10. The veterinarian DR browses the health management screen 100 through the doctor terminal 11 and uses the screen as a reference for the medical care.

In FIG. 2, the medical care information 15 includes owner information 16 that is information relating to the owner OW and pet information 17 that is information about the pet PT. The owner information 16 includes owner basic information 18 and precaution information 19. The pet information 17 includes pet basic information 20, medical record information 21, character information 22, breeding environment information 23, pet food information 24, and medication information 25.

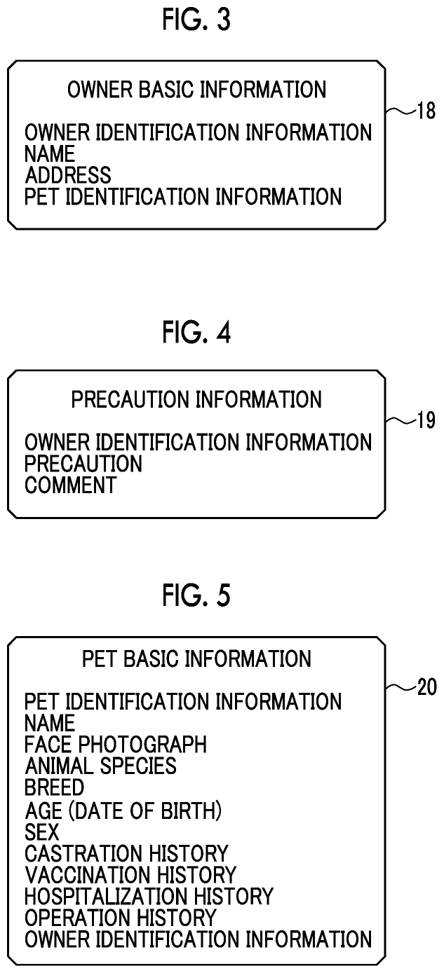

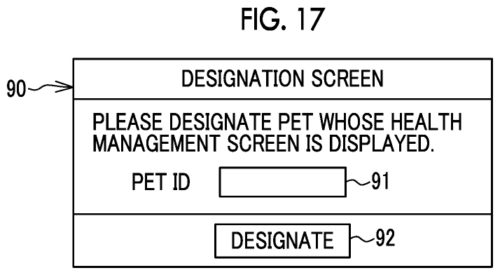

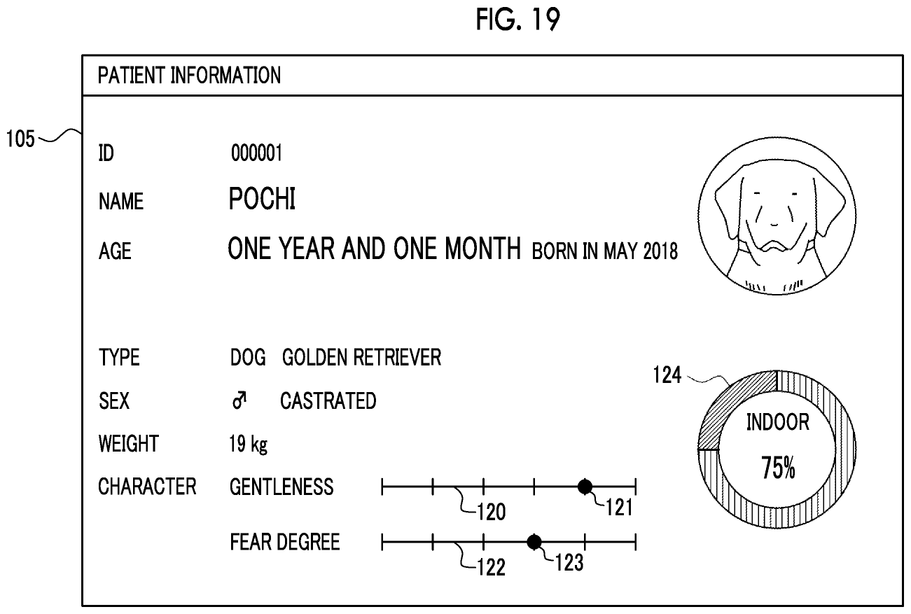

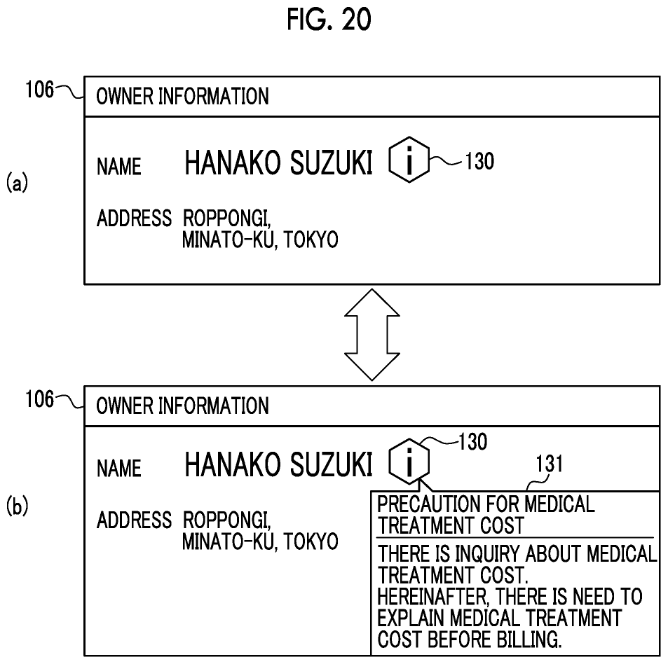

As shown in FIG. 3, owner identification information for identifying individual owners OW, a name and an address of an owner OW, and pet identification information for identifying the pet PT owned by the owner OW are registered in the owner basic information 18. The owner identification information and the pet identification information are expressed by a combination of a plurality of symbols and/or numbers. In a case where the owner OW has a plurality of pets PT, that is, is a so-called multiple-animal owner and the plurality of pets PT receive the medical care at the animal hospital AH, the pet identification information for the plurality of pets is registered in the owner basic information 18. The owner basic information 18 is registered at the time of the first medical care of the pet PT or the like. The pet identification information may be denoted as “pet identification data (ID)” (refer to FIG. 17) or simply “ID” (refer to FIG. 19).

As shown in FIG. 4, the owner identification information, a precaution, and a comment are registered in the precaution information 19. The precaution relates to handling of the owner OW. The comment is a supplementary explanation of the precaution or the like. There is a case where the registration is made only for the precaution, not for the comment. Further, there is a case where the precaution information 19 itself does not exist depending on the owner OW.

As shown in FIG. 5, pet identification information, the name, face photograph, animal species, breed, age (date of birth), sex, castration history, vaccination history, hospitalization history, and operation history, and owner identification information of the pet PT are registered in the pet basic information 20. The animal species is a dog, a cat, and the like. In a case where the animal species is the dog, the breed is Yorkshire Terrier, Papillon, Maltese, Labrador Retriever, Shiba, or the like. In a case where the animal species is the cat, the breed is Ragdoll, Himalayan, Ragamuffin, or the like. Most of the pet basic information 20 such as the name, animal species, breed, age, and sex are registered at the time of the first medical care of the pet PT, similar to the owner basic information 18. The castration history, the vaccination history, the hospitalization history, and the operation history are registered each time such an event occurs.

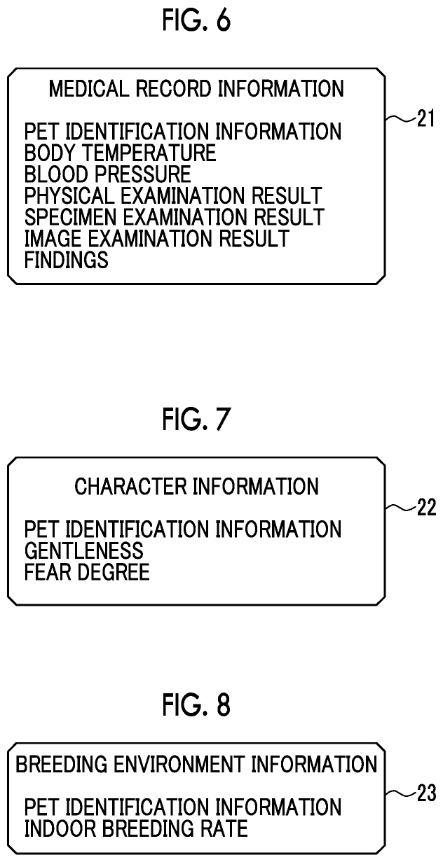

As shown in FIG. 6, the pet identification information, the body temperature, blood pressure, physical examination result, specimen examination result, and image examination result of the pet PT, and findings of the veterinarian DR are registered in the medical record information 21. The physical examination result includes body height, body length, weight, body fat percentage, and the like. The specimen examination result includes blood examination result, urine examination result, biochemical examination result, and the like. The blood examination result includes the number of red blood cells, the number of white blood cells, hemoglobin, and the like. The urine examination result includes urine protein, urine sugar, urinary occult blood, urobilinogen, and the like. The biochemical examination result includes albumin, creatinine, total cholesterol, and the like. The image examination result is an X-ray image which is an X-ray examination result, an endoscope image which is an endoscopic examination result, an ultrasound image which is an ultrasound examination result, and the like. The body temperature, blood pressure, physical examination result, specimen examination result, image examination result, and findings are registered in a time series.

As shown in FIG. 7, the pet identification information, gentleness, and a fear degree are registered in the character information 22. The gentleness is evaluated on a six-level scale of levels one to six (refer to FIG. 19), and a more moderate character is at a higher level. The fear degree is evaluated on a six-level scale of levels one to six (refer to FIG. 19) as in the gentleness, and a more feared character is at a higher level. The character information 22 is registered at the time of the first medical care of the pet PT.

As shown in FIG. 8, the pet identification information and an indoor breeding rate are registered in the breeding environment information 23. The indoor breeding rate is a rate at which the pet PT is bred indoors literally, and is expressed in %. In a case where the pet PT is always kept indoors, the indoor breeding rate is 100%.

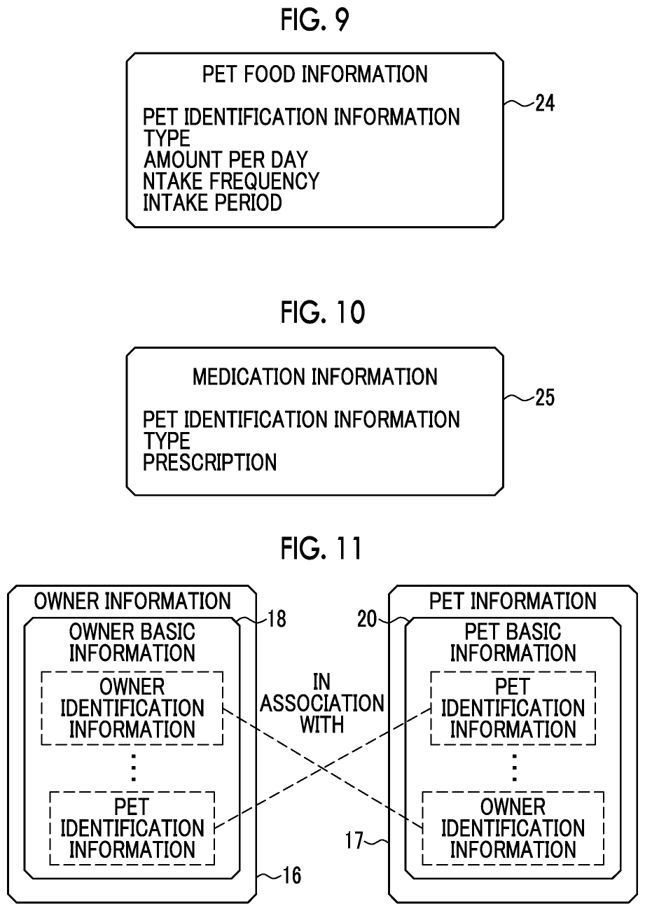

As shown in FIG. 9, the pet identification information, a type of pet food given to the pet PT, an amount per day, an intake frequency, and an intake period are registered in the pet food information 24. As shown in FIG. 10, the pet identification information, a type of medicine administered to the pet PT, and a prescription are registered in the medication information 25.

As shown in FIG. 3, the pet identification information is registered in the owner basic information 18 in addition to the owner identification information. As shown in FIG. 5, the owner identification information is registered in the pet basic information 20 in addition to the pet identification information. Therefore, as shown in FIG. 11, the owner information 16 and the pet information 17 are associated with each other by the owner identification information and the pet identification information.

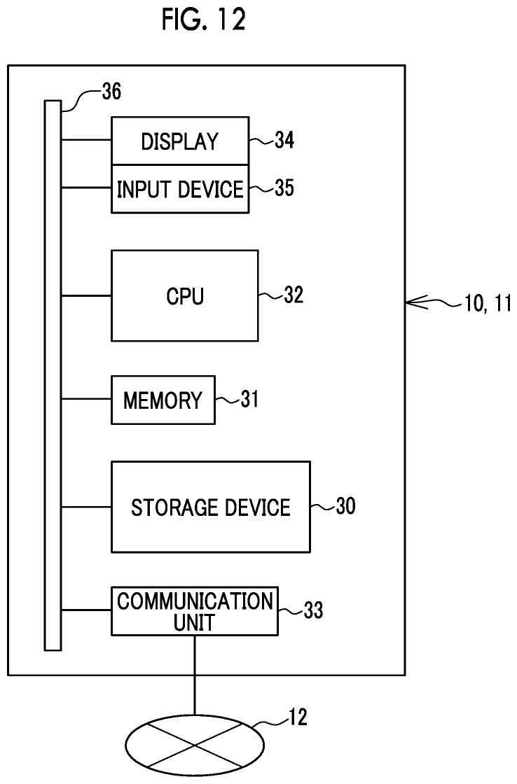

In FIG. 12, the computers constituting the medical care support server 10 and the doctor terminal 11 have basically the same configuration and comprise a storage device 30, a memory 31, a central processing unit (CPU) 32, a communication unit 33, a display 34, and an input device 35. These are connected to each other through a bus line 36.

The storage device 30 is a hard disk drive built in the computers constituting the medical care support server 10 and the doctor terminal 11, or connected through a cable or a network. Alternatively, the storage device 30 is a disk array in which a plurality of hard disk drives are connected. The storage device 30 stores a control program such as an operating system, various application programs (hereinafter abbreviated as application program (AP)), various pieces of data accompanying these programs, and the like. A solid state drive may be used instead of the hard disk drive.

The memory 31 is a work memory for the CPU 32 to execute processing. The CPU 32 loads the program stored in the storage device 30 into the memory 31 and executes processing according to the program to integrally control each part of the computer.

The communication unit 33 is a network interface that controls transmission of various pieces of information through the network 12. The display 34 displays various screens. The various screens are provided with an operation function using a graphical user interface (GUI). The computers constituting the medical care support server 10 and the doctor terminal 11 receive an input of an operation instruction from the input device 35 through various screens. The input device 35 is a keyboard, a mouse, a touch panel, or the like.

In the following description, a suffix “A” is assigned to each part of the computer constituting the medical care support server 10 and a suffix “B” is assigned to each part of the computer constituting the doctor terminal 11 as signs to distinguish the computers.

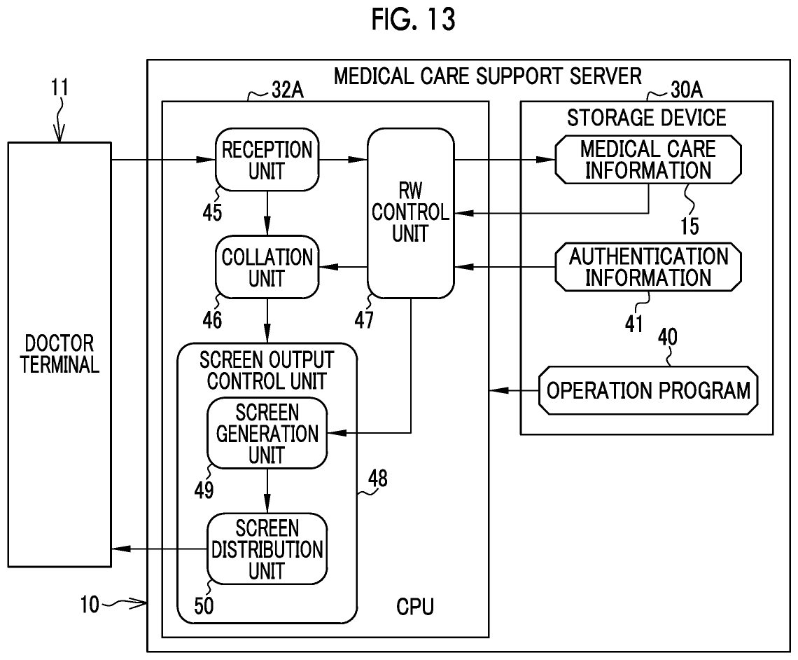

In FIG. 13, an operation program 40 is stored in the storage device 30A of the medical care support server 10. The operation program 40 is an AP for causing the computer to function as the medical care support server 10. That is, the operation program 40 is an example of an “operation program of medical care support device” according to the technique of the present disclosure. The storage device 30A stores the medical care information 15 and authentication information 41 in addition to the operation program 40. A plurality of pieces of medical care information 15 are stored for each owner OW and pet PT.

In a case where the operation program 40 is activated, the CPU 32A of the medical care support server 10 cooperates with the memory 31 and the like to function as a reception unit 45, a collation unit 46, and a read/write (hereinafter abbreviated as read/write (RW)) control unit 47, and a screen output control unit 48. The screen output control unit 48 has a screen generation unit 49 and a screen distribution unit 50. The “screen output control unit 48” described below is a concept including the screen generation unit 49 and the screen distribution unit 50.

The reception unit 45 receives the medical care information 15 such as the precaution information 19 transmitted from the doctor terminal 11. The reception unit 45 outputs the medical care information 15 to the RW control unit 47.

The reception unit 45 receives doctor identification information and a password transmitted from the doctor terminal 11. The doctor identification information and the password are information for the veterinarian DR to obtain authentication of access permission to the medical care support server 10. The reception unit 45 outputs the received doctor identification information and password to the collation unit 46. The doctor identification information may be denoted as “user identification data (ID)” (refer to FIG. 15).

Further, the reception unit 45 receives the request for distributing the health management screen 100 transmitted from the doctor terminal 11. The reception unit 45 outputs the distribution request to the RW control unit 47.

The collation unit 46 receives the doctor identification information and the password from the reception unit 45. The collation unit 46 collates the received doctor identification information and password with doctor identification information and a password registered in advance as the authentication information 41. The collation unit 46 outputs the collation result of the doctor identification information and the password to the screen output control unit 48.

The RW control unit 47 controls storage of the various pieces of information in the storage device 30A and reading of the various pieces of information in the storage device 30A. The RW control unit 47 stores the medical care information 15 such as the precaution information 19 in the storage device 30A. That is, the RW control unit 47 is an example of a “storage control unit” according to the technique of the present disclosure. The RW control unit 47 stores the authentication information 41 in the storage device 30A.

The RW control unit 47 reads out the medical care information 15 corresponding to the distribution request from the reception unit 45 from the storage device 30A and outputs the read medical care information 15 to the screen generation unit 49 of the screen output control unit 48. Further, the RW control unit 47 reads out the authentication information 41 from the storage device 30A and outputs the read authentication information 41 to the collation unit 46.

The screen output control unit 48 controls the output of various screens such as the health management screen 100 to the doctor terminal 11. More specifically, the screen output control unit 48 outputs the various screens in a form of, for example, screen data for web distribution created in a markup language such as extensible markup language (XML). Accordingly, it is possible to browse the various screens on a web browser through the doctor terminal 11. Instead of XML, another data description language such as JavaScript (registered trademark) Object Notation (JSON) may be used.

The screen generation unit 49 generates screen data of the various screens and outputs the generated screen data to the screen distribution unit 50. The screen distribution unit 50 distributes the screen data from the screen generation unit 49 to the doctor terminal 11.

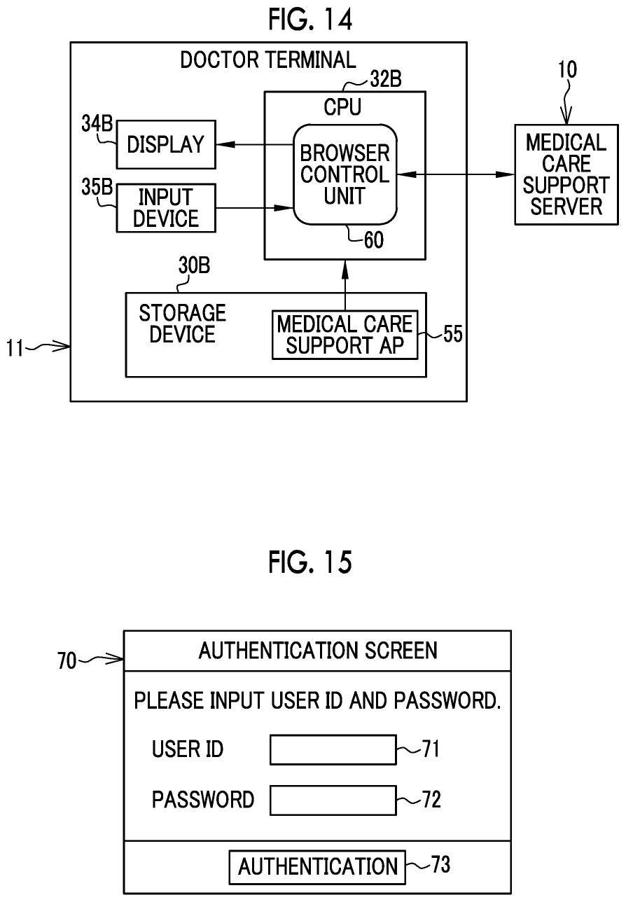

In FIG. 14, medical care support AP 55 is stored in the storage device 30B of the doctor terminal 11. In a case where the medical care support AP 55 is executed and the web browser is activated, the CPU 32B of the doctor terminal 11 cooperates with the memory 31 and the like to function as a browser control unit 60. The browser control unit 60 controls the operation of the web browser. The browser control unit 60 receives the screen data of the various screens from the medical care support server 10. The browser control unit 60 reproduces the various screens to be displayed on the web browser based on the screen data and displays the various screens on the display 34B.

The browser control unit 60 receives various operation instructions to be input from the input device 35B by the veterinarian DR through the various screens. The operation instructions include an authentication instruction for the access permission to the medical care support server 10, an input instruction and a transmission instruction for the medical care information 15, a distribution instruction for the health management screen 100, and the like. The browser control unit 60 issues a request in response to the operation instruction to the medical care support server 10. For example, the browser control unit 60 issues the request for distributing the health management screen 100 to the medical care support server 10 in response to the distribution instruction for the health management screen 100.

The various screens 70, 80, 90, 100, and the like described below are screens output by the screen output control unit 48 of the medical care support server 10 and displayed on the display 34B by the browser control unit 60 of the doctor terminal 11.

FIG. 15 shows an authentication screen 70 for performing the authentication instruction for the access permission to the medical care support server 10. The authentication screen 70 is provided with an input box 71 for the doctor identification information (denoted as user ID in FIG. 15) and an input box 72 for the password. The authentication screen 70 is provided with an authentication button 73.

In a case where the doctor identification information is input to the input box 71, the password is input to the input box 72, and then the authentication button 73 is selected, the input doctor identification information and password are transmitted from the doctor terminal 11 to the medical care support server 10.

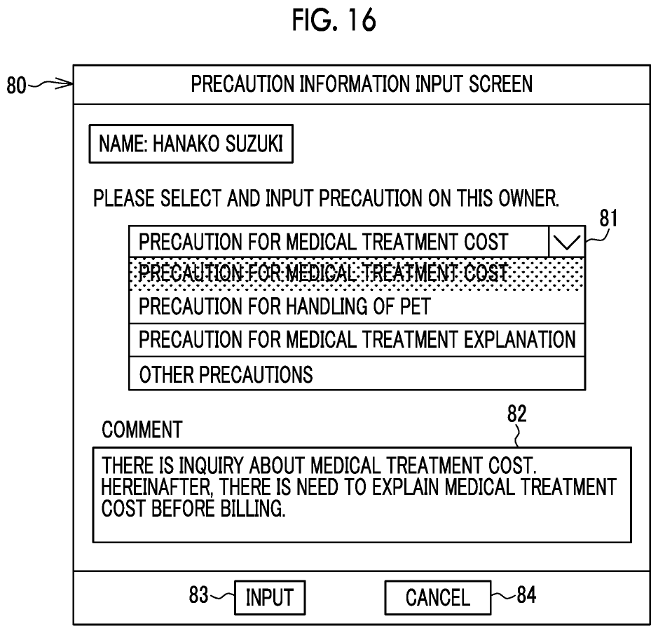

FIG. 16 shows a precaution information input screen 80 for performing the input instruction and the transmission instruction of precaution information 19. The precaution information input screen 80 is provided with a pull-down menu 81 for inputting the precautions and a comment input box 82. Further, the precaution information input screen 80 is provided with an input button 83 and a cancel button 84.

Four options of “precaution for medical treatment cost”, “precaution for handling of pet”, “precaution for medical treatment explanation”, and “other precautions” are prepared in advance in the pull-down menu 81. One of these four options can be selected in the pull-down menu 81. FIG. 16 shows a state where “precaution for medical treatment cost” is selected as the precaution, as indicated by hatching. FIG. 16 shows a state where “There is an inquiry about the medical treatment cost. Hereinafter, there is a need to explain the medical treatment cost before billing” is input to the input box 82 as the comment.

In a case where one of the options in the pull-down menu 81 is selected and a comment is input to the input box 82 in some cases, and then the input button 83 is selected, the selected precautions and the input comment in some cases are transmitted from the doctor terminal 11 to the medical care support server 10 as the precaution information 19. In a case where the cancel button 84 is selected, the display of the precaution information input screen 80 is erased and thus the input of the precaution information 19 is stopped.

Although illustration is omitted, input screens for the various pieces of information input screens such as an input screen for the owner basic information 18, an input screen for the pet basic information 20, and an input screen for the medical record information 21 are output from the screen output control unit 48 and displayed on the display 34B, in addition to the precaution information input screen 80. The various pieces of information are input through the input screens.

FIG. 17 shows a designation screen 90 for performing the distribution instruction for the health management screen 100. The designation screen 90 is provided with an input box 91 for the pet identification information (denoted as pet ID in FIG. 17). The designation screen 90 is provided with a designation button 92.

In a case where the pet identification information of the pet PT displayed on the health management screen 100 is input to the input box 91 and then the designation button 92 is selected, a distribution request including the input pet identification information is transmitted from the doctor terminal 11 to the medical care support server 10.

In FIG. 18, the health management screen 100 is roughly divided into a first section 101, a second section 102, and a third section 103. The first section 101 occupies a left side portion of the health management screen 100, the second section 102 occupies a central portion of the health management screen 100, and the third section 103 occupies a right portion of the health management screen 100, respectively. The first section 101 has a first display region 105, a second display region 106, and a third display region 107. The second section 102 has a fourth display region 108. The third section 103 has a fifth display region 109, a sixth display region 110, a seventh display region 111, an eighth display region 112, and a ninth display region 113.

In FIG. 19, the first display region 105 displays the pet identification information (denoted as ID in FIG. 19), the name, face photograph, age, animal species and breed (collectively denoted as type in FIG. 19), sex, castration history (collectively denoted as sex in FIG. 19), and weight of the pet PT. The first display region 105 also displays a bar 120 and a mark 121 indicating the level of gentleness and a bar 122 and a mark 123 indicating the level of fear degree of the character information 22. The bars 120 and 122 have “level 1” at left ends, and “level 6” at right ends. Further, the first display region 105 displays the indoor breeding rate of the breeding environment information 23 and an annular graph 124 indicating the indoor breeding rate.

FIG. 19 illustrates a pet PT having pet identification information “000001”, name “Pochi”, age “one year and one month”, animal species and breed “dog and Golden Retriever”, sex and castration history “♂ (male) and castrated”, weight “19 kg”, gentleness “level 5”, fear degree “level 4”, and indoor breeding rate “75%”.

In (a) and (b) of FIG. 20, the name and the address of the owner OW are displayed in the second display region 106. In a case where the precaution information 19 is present, an information mark 130 indicating the presence of the precaution information 19 is displayed in the second display region 106. That is, the information mark 130 is an example of a “mark indicating presence of the precaution information” according to the technique of the present disclosure.

The information mark 130 is selectable. While the information mark 130 is not selected by the veterinarian DR, only the information mark 130 is displayed in the second display region 106 as shown in (a) of FIG. 20. In this state, in a case where the information mark 130 is selected by the veterinarian DR, a balloon display region 131 appears below the information mark 130 as shown in (b) of FIG. 20. The precaution information 19 is displayed in the balloon display region 131. The balloon display region 131 is an example of a “display region dedicated to precaution information” according to the technique of the present disclosure. An instruction to select the information mark 130 in a state where the balloon display region 131 is not displayed as in (a) of FIG. 20 is an example of an “instruction to display precaution information” according to the technique of the present disclosure.

In a case where the information mark 130 is selected again in a state where the balloon display region 131 is displayed as in (b) of FIG. 20, the state is returned to the state where the balloon display region 131 is not displayed as in (a) of FIG. 20. That is, the states of (a) and (b) of FIG. 20 can be switched by the instruction to select the information mark 130.

(a) and (b) of FIG. 20 illustrate an owner OW having a name “Hanako Suzuki” and an address “Roppongi, Minato-ku, Tokyo”. (a) and (b) of FIG. 20 also illustrate a case where the same precautions and comments as those shown in FIG. 16 are displayed as the precaution information 19.

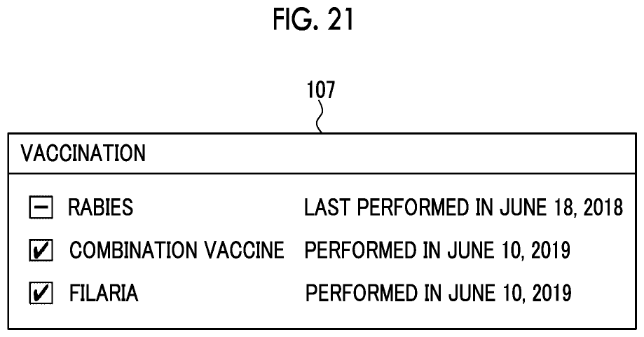

In FIG. 21, the vaccination history is displayed in the third display region 107. FIG. 21 illustrates a vaccination history that rabies vaccination has not been performed since June 18, 2018 and a combination vaccine and filaria vaccination have been performed in June 10, 2019. A valid period of an inoculated vaccine, the remaining number of inoculations, and the like may be added to the display of the vaccination history.

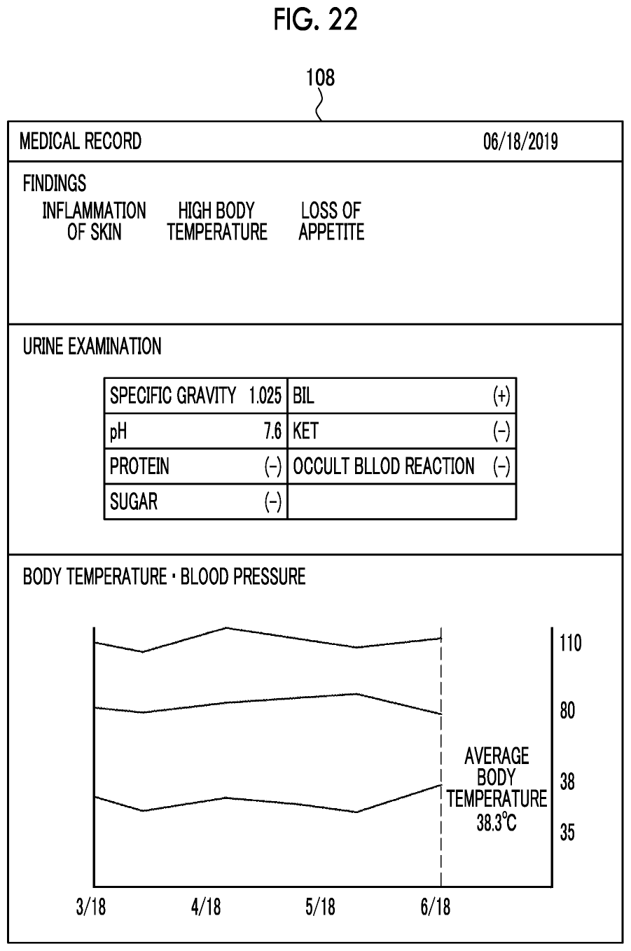

In FIG. 22, the medical record information 21 is extracted and displayed in the fourth display region 108. FIG. 22 shows an example in which findings, urine examination result, body temperature, and blood pressure are extracted and displayed. Contents of the medical record information 21 to be extracted and displayed can be changed by the veterinarian DR.

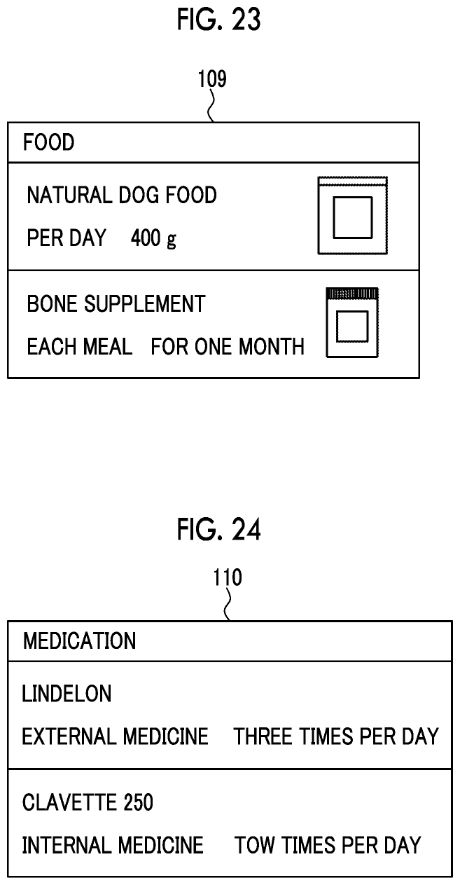

In FIG. 23, the pet food information 24 is displayed in the fifth display region 109. FIG. 23 illustrates the pet food information 24 indicating that 400 g of natural dog food is provided per day and bone supplement is provided for one month each meal.

In FIG. 24, the medication information 25 is displayed in sixth display region 110. FIG. 24 illustrates the medication information 25 indicating that Lindelon (registered trademark) is being administered three times a day and Clavette 250 is being administered twice a day.

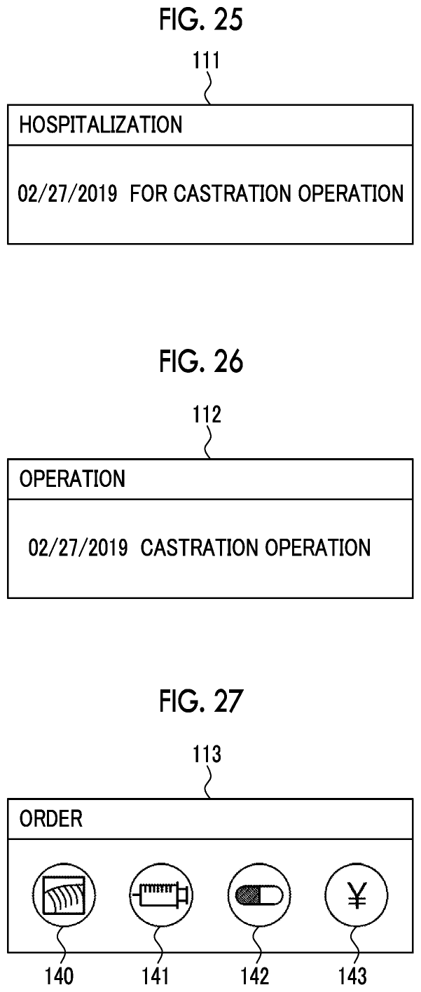

In FIG. 25, the hospitalization history is displayed in the seventh display region 111. FIG. 25 illustrates the hospitalization history indicating that the hospitalization is made for a castration operation in Feb. 10, 2019.

In FIG. 26, the operation history is displayed in the eighth display region 112. FIG. 26 illustrates the operation history indicating that castration is performed in Feb. 10, 2019.

In FIG. 27, the ninth display region 113 is provided with a first request button 140, a second request button 141, a third request button 142, and a fourth request button 143 for requesting various orders. The first request button 140 is a button for requesting an image examination. The second request button 141 is a button for requesting a specimen examination. The third request button 142 is a button for requesting preparation of a medicine to be prescribed. The fourth request button 143 is a button for requesting an estimation of the medical treatment cost.

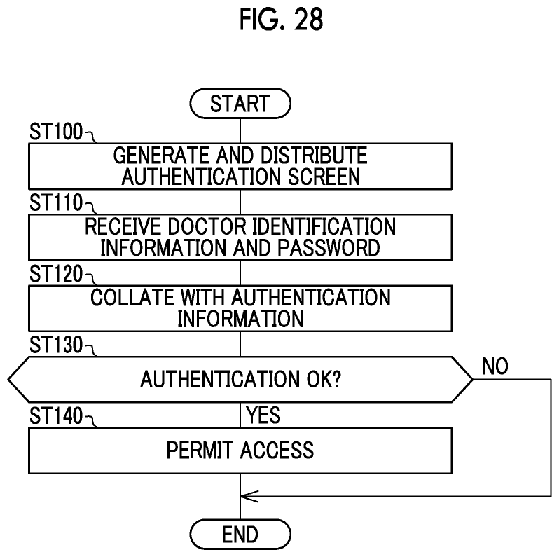

Next, the action of the above configuration will be described with reference to flowcharts of FIGS. 28 to 30. First, in a case where the operation program 40 is activated, the CPU 32A of the medical care support server 10 functions as the reception unit 45, the collation unit 46, the RW control unit 47, and the screen output control unit 48 (screen generation unit 49 and screen distribution unit 50), as shown in FIG. 13.

In a case where the medical care support AP 55 is activated in the doctor terminal 11, the screen generation unit 49 generates the authentication screen 70 shown in FIG. 15 in the medical care support server 10. The generated authentication screen 70 is distributed to the doctor terminal 11 by the screen distribution unit 50 (step ST100 in FIG. 28).

In the doctor terminal 11, the authentication screen 70 is displayed on the display 34B by the browser control unit 60. The veterinarian DR inputs the doctor identification information into the input box 71 and the password into the input box 72, and selects the authentication button 73. This causes the input doctor identification information and password to be transmitted from the doctor terminal 11 to the medical care support server 10.

In the medical care support server 10, the doctor identification information and the password from the doctor terminal 11 are received by the reception unit 45 (step ST110). The doctor identification information and the password are output from the reception unit 45 to the collation unit 46.

The collation unit 46 collates the doctor identification information and the password from the reception unit 45 with the doctor identification information and the password registered in advance in the storage device 30A as the authentication information 41 (step ST120). As a result of the collation, in a case where the doctor identification information and the password from the reception unit 45 match any of the doctor identification information and the password registered in advance as the authentication information 41, access of the veterinarian DR to the medical care support server 10 is permitted (YES in step ST130, step ST140). In a case where the access to the medical care support server 10 is permitted, the input instruction and the transmission instruction for the medical care information 15 including the precaution information 19 can be performed. In addition, in the case where the access to the medical care support server 10 is permitted, the request for distributing the health management screen 100 can be performed.

On the other hand, in a case where the doctor identification information and the password from the reception unit 45 do not match any of the doctor identification information and the password registered in advance as the authentication information 41, the access of the veterinarian DR to the medical care support server 10 is denied (NO in step ST130). In this case, a screen displaying a message such as “authentication failed” is output from the screen output control unit 48 and displayed on the display 34B.

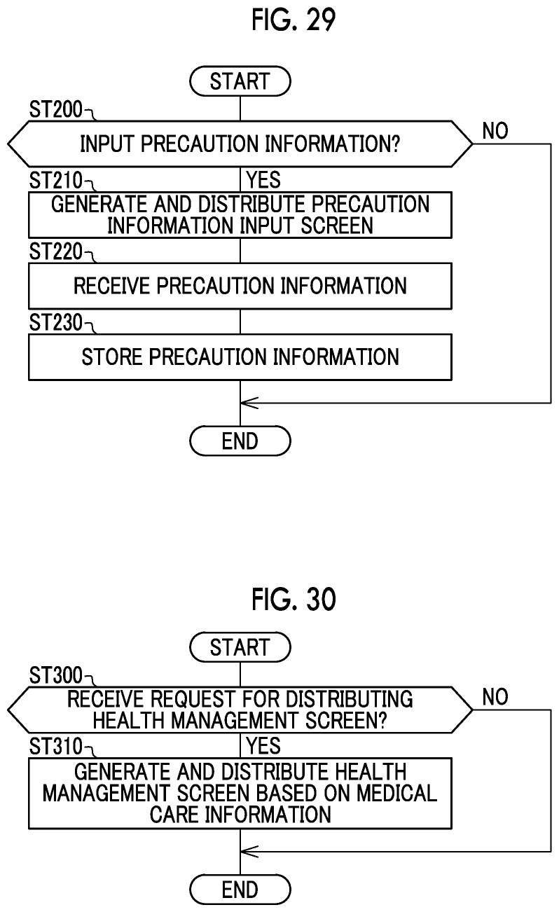

In FIG. 29, in a case where there is an instruction to input the precaution information 19 from the veterinarian DR permitted to access the medical care support server 10 through the doctor terminal 11 (YES in step ST200), the screen generation unit 49 generates the precaution information input screen 80 shown in FIG. 16 in the medical care support server 10. The generated precaution information input screen 80 is distributed to the doctor terminal 11 by the screen distribution unit 50 (step ST210).

In the doctor terminal 11, the browser control unit 60 displays the precaution information input screen 80 on the display 34B. The veterinarian DR selects one precaution from the plurality of options in the pull-down menu 81, inputs a comment in the input box 82 in some cases, and selects the input button 83. This causes the selected precaution and the input comment in some cases to be transmitted from the doctor terminal 11 to the medical care support server 10 as the precaution information 19.

In the medical care support server 10, the precaution information 19 from the doctor terminal 11 is received by the reception unit 45 (step ST220). The precaution information 19 is output from the reception unit 45 to the RW control unit 47. The precaution information 19 is stored in the storage device 30A by the RW control unit 47 (step ST230). Step ST220 is an example of a “reception step” according to the technique of the present disclosure. Step ST230 is an example of a “storage control step” according to the technique of the present disclosure.

In FIG. 30, in a case where there is the request for distributing the health management screen 100 from the veterinarian DR permitted to access the medical care support server 10 through the designation screen 90 shown in FIG. 17 and the distribution request is received by the reception unit 45 (YES in step ST300), the RW control unit 47 reads out the medical care information 15 having the pet identification information included in the distribution request from the storage device 30A in the medical care support server 10. The read medical care information 15 is output from the RW control unit 47 to the screen output control unit 48. In the screen output control unit 48, the screen generation unit 49 generates the health management screen 100 shown in FIG. 18 based on the medical care information 15. The generated health management screen 100 is distributed to the doctor terminal 11 by the screen distribution unit 50 (step ST310). Step ST310 is an example of a “screen output control step” according to the technique of the present disclosure.

In the doctor terminal 11, the browser control unit 60 displays the health management screen 100 on the display 34B. The veterinarian DR performs medical care with reference to the health management screen 100.

As shown in (b) of FIG. 20, the balloon display region 131 of the precaution information 19 is displayed in the second display region 106 of the health management screen 100. The balloon display region 131 is not normally displayed as shown in (a) of FIG. 20, and only the information mark 130 is displayed in the second display region 106. In this state, the balloon display region 131 is displayed only in a case where there is the instruction to select the information mark 130.

As described above, the medical care support server 10 comprises the reception unit 45, the RW control unit 47 as the storage control unit, and the screen output control unit 48. The reception unit 45 receives the precaution information 19 on the owner OW, which is input by the veterinarian DR, as the owner information 16. The RW control unit 47 performs control to store the precaution information 19 in the storage device 30A. The screen output control unit 48 performs control to output the health management screen 100 provided with the balloon display region 131 which is the display region dedicated to the precaution information 19. By providing the balloon display region 131, it is possible to prevent the precaution information 19 from being buried in other pieces of information and thus to prevent the precaution information 19 from being overlooked.

In a case where there are a plurality of veterinarians DR at the animal hospital AH, other veterinarians DR other than the veterinarian DR who has input the precaution information 19 can also share the information by viewing the balloon display region 131. It is possible to avoid trouble due to erroneous responding to the owner OW by other veterinarians DR.

The screen output control unit 48 displays the balloon display region 131 only in a case where there is the instruction to select the information mark 130 which is the instruction to display the precaution information 19. Therefore, in a situation where the owner OW cannot browse the health management screen 100, such as a pre-examination before medical care, it is possible to check the precaution information 19 with the display of the balloon display region 131. Conversely, in a situation where the owner OW can browse the health management screen 100, such as the medical treatment explanation at the medical care using the health management screen 100, it is possible to perform non-display of the balloon display region 131. Therefore, it is possible to effectively keep the precaution information 19 confidential from the owner OW.

In a state where the balloon display region 131 is not displayed, the screen output control unit 48 displays the information mark 130 indicating the presence of the precaution information 19 in the second display region 106 of the health management screen 100. Therefore, it is possible for the veterinarian DR to view at a glance that the precaution information 19 is present. On the contrary, the owner OW does not know a meaning of the information mark 130. Therefore, even in a case where the owner OW browses the health management screen 100, it is still possible to keep the precaution information 19 confidential.

The precaution of the precaution information 19 is input by being selected from the plurality of options prepared in advance. It is possible to save labor of inputting the precaution information 19 as compared with a case where the precaution is input in a free word. In the case of the free word, expressions may fluctuate even with the same contents. However, there is no such concern in the invention.

The reception unit 45 receives the pet information 17 as well as the owner information 16. As shown in FIG. 11, the RW control unit 47 performs control to store the owner information 16 and the pet information 17 in association with each other. The screen output control unit 48 displays the owner information 16 and the pet information 17 on the health management screen 100. Therefore, it is possible to collect and display information necessary for medical care on the health management screen 100.

The health management screen 100 is mainly composed of the first section 101 on the left side grouped by the owner basic information 18 and the pet basic information 20, the central second section 102 grouped by the medical record information 21, and the third section 103 on the right grouped by the pet food information 24, the medication information 25, the request buttons 140 to 143 for various orders, and the like. Therefore, it is possible for the veterinarian to naturally obtain information along a flow of the medical care by moving the eyes from the first section 101 to the second section 102 and from the second section 102 to the third section 103.

As shown in FIG. 2, the pet information 17 includes the medical record information 21, the character information 22, the breeding environment information 23, and the like. With the medical record information 21, it is possible to grasp a health state of the pet PT. With the character information 22, it is possible to grasp the character of the pet PT. It is possible for the veterinarian DR to perform the medical care that is adapted to the grasped character of the pet PT, such as increasing the number of people who hold limbs of a pet PT in X-ray examination for the pet PT with a relatively low level of gentleness or performing the medical care with great care so as not to scare a pet PT for the pet PT with a relatively high level of fear degree. As a result, it is possible to improve the medical care efficiency. For example, in a case of a pet PT having a relatively low indoor breeding rate, it is possible to use the breeding environment information 23 for specifying an infectious disease, such as suspecting an airborne infectious disease.

There is a browsing restriction on the health management screen 100 by authentication, and the screen output control unit 48 outputs the health management screen 100 only in a case where the authentication is permitted. Therefore, it is possible to further improve the confidentiality of the precaution information 19.

In a case where the “precaution for medical treatment cost” is selected as the precaution, the estimation of the medical treatment cost may be requested without waiting for the selection of the fourth request button 143 by the veterinarian DR. For example, in a case where an abnormal value is found in a numerical value of the specimen examination in the medical record information 21, display may be performed to prompt the veterinarian DR to request another examination order related to the numerical value in which the abnormal value is found.

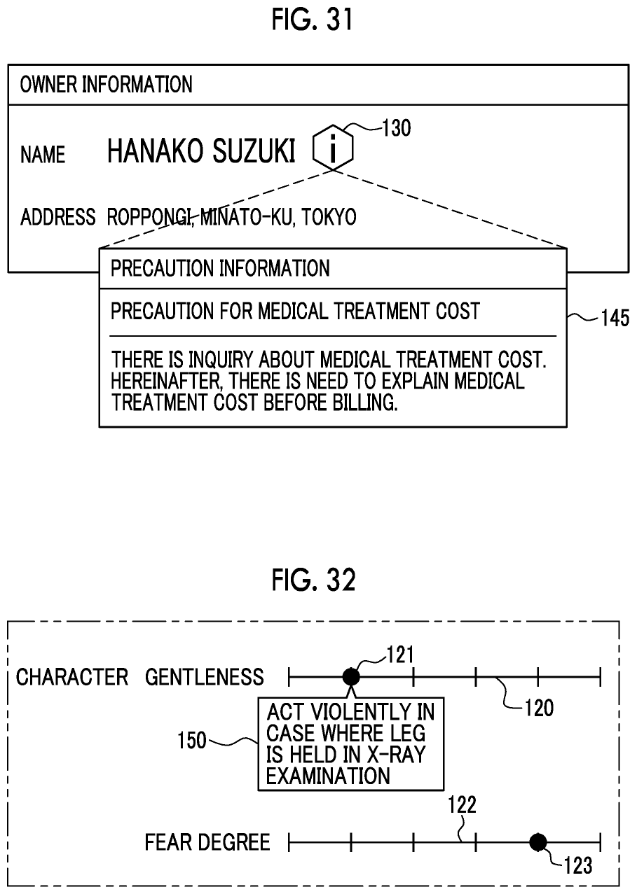

The display region dedicated to the precaution information 19 is not limited to the balloon display region 131 illustrated in (b) of FIG. 20. For example, a pop-up screen 145 that appears in a case where the information mark 130 is selected may be the display region dedicated to the precaution information 19 as shown in FIG. 31. The pop-up screen 145 is a part of the health management screen 100.

In a case where the animal species is a dog, it is said that the character of the owner OW has a rough tendency depending on the breed of the kept pet PT. A display order of the precautions in the pull-down menu 81 may be changed according to the breed of the pet PT. For example, the option of “precaution for handling of pet” is displayed at the top for an owner OW who keeps a breed that has a strong tendency to be doted on the pet PT. Alternatively, the options of the precautions in the pull-down menu 81 may be changed according to the breed of the pet PT. As described above, in a case where the display order of the precautions or the precaution itself is changed according to the breed of the pet PT, it is possible for the veterinarian DR to easily select the precaution.

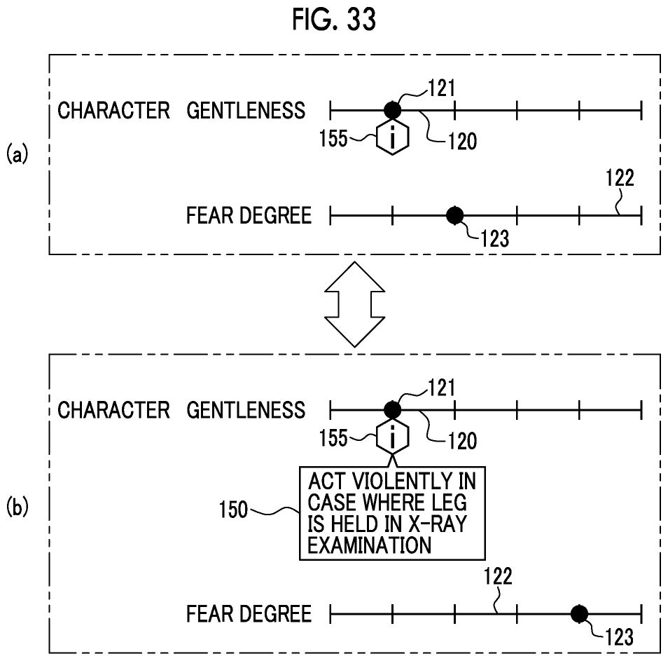

The comment may be composed to be registrable in the character information 22 as in the precaution information 19. In this case, it is preferable to display the comment in the balloon display region 150 as shown in FIG. 32. Alternatively, as shown in (a) of FIG. 33, only the information mark 155 is normally displayed and the balloon display region 150 may be displayed as shown in (b) of FIG. 33 only in a case where the information mark 155 is selected, similar to the precaution information 19. FIGS. 32 and 33 illustrate a case where the comment is “act violently in a case where a leg is held in X-ray examination”. With the display of the comment in this manner, it is possible to immediately check a reason for evaluating the character of the pet PT.

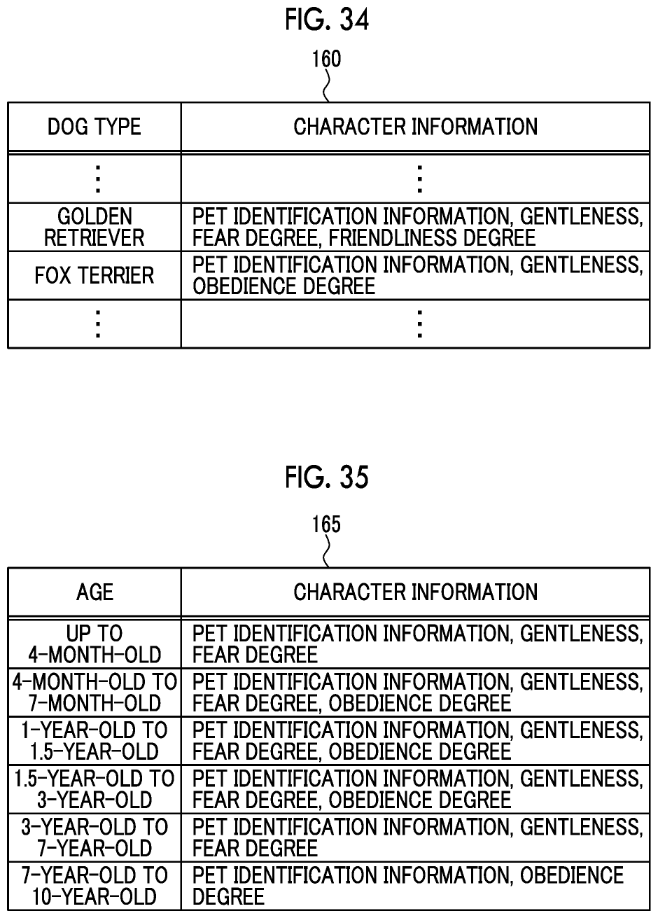

As shown in table 160 of FIG. 34, the character to be registered in the character information 22 may be changed according to the breed. FIG. 34 shows an example in which friendliness degree in a case where the breed is Golden Retriever and an obedience degree in a case where the breed is Fox Terrier are respectively added to the gentleness and the fear degree in the first embodiment.

It is said that the character of the pet PT has a rough tendency depending on the breed. Therefore, in a case where the character to be registered in the character information 22 is changed according to the breed, it is possible to obtain the character information 22 suitable for the breed as shown in FIG. 34.

As shown in table 165 of FIG. 35, the character to be registered in the character information 22 may be changed according to the age. FIG. 35 shows an example in which the same gentleness and fear degree as in the first embodiment in a case where the age is up to 4-month-old and from 3-year-old to 7-year-old, the obedience degree in addition to the gentleness and the fear degree in a case where the age is from 4-month-old to 7-month-old, 1-year-old to 1.5-year-old, and 1.5-year-old to 3-year-old, and only the fear degree in a case where the age is 7-year-old to 10-year-old are respectively registered.

The character of the pet PT also changes with the age. Therefore, in a case where the character to be registered in the character information 22 is changed according to the age, it is possible to obtain the character information 22 suitable for the age as shown in FIG. 35. The embodiment shown in FIG. 34 and the embodiment shown in FIG. 35 may be implemented in combination, and the character to be registered in the character information 22 may be changed according to the breed and the age.

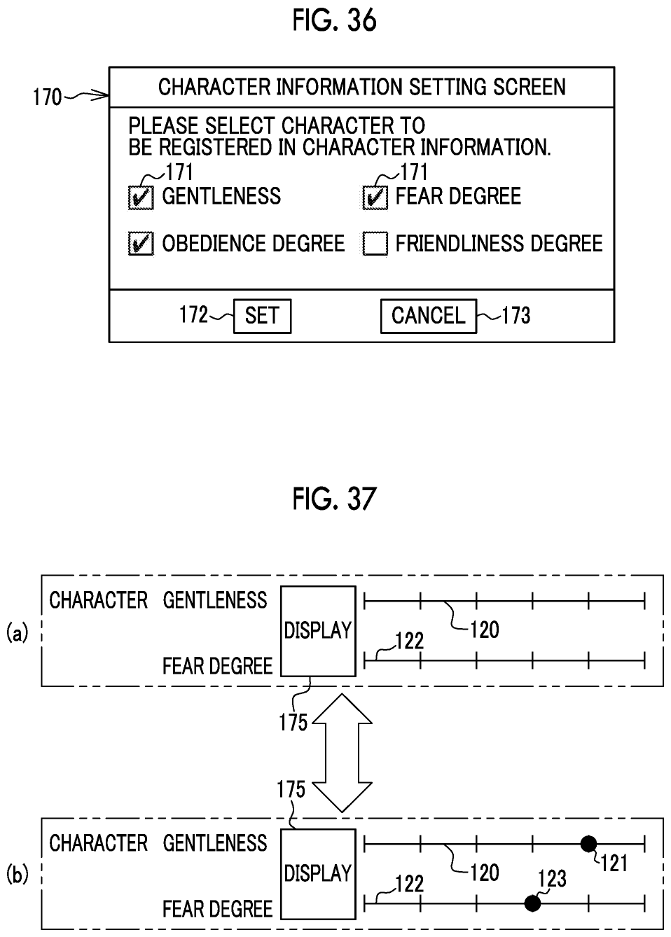

For example, the character to be registered in the character information 22 may be composed to be selectable using a character information setting screen 170 shown in FIG. 36.

In FIG. 36, check boxes 171 are respectively provided for the gentleness, the fear degree, the obedience degree, and the friendliness degree on the character information setting screen 170. Further, the character information setting screen 170 is provided with a setting button 172 and a cancel button 173.

In a case where the setting button 172 is selected, the character whose check box 171 is checked at that time is set as the character to be registered in the character information 22. FIG. 36 shows an example in which the check boxes 171 of the gentleness, the fear degree, the obedience degree are checked. In a case where the cancel button 173 is selected, the display of the character information setting screen 170 is erased, and the character setting is stopped.

As described above, in a case where the character to be registered in the character information 22 is composed to be selectable, it is possible for the veterinarian DR to freely customize the character to be registered in the character information 22.

### Second Embodiment

In a second embodiment shown in (a) and (b) of FIG. 37, the character information 22 is switched between display and non-display.

As shown in (a) and (b) of FIG. 37, in the second embodiment, a display switching button 175 is provided on a left lateral side of the bars 120 and 122.

(a) of FIG. 37 shows a state where only the bars 120 and 122 are displayed and the marks 121 and 123 are not displayed. On the other hand, (b) of FIG. 37 shows a state where the marks 121 and 123 are displayed. The states of (a) and (b) of FIG. 37 are switched by an instruction to select the display switching button 175. That is, the instruction to select the display switching button 175 in the state where the marks 121 and 123 in (a) of FIG. 37 are not displayed is an example of an “instruction to display character information” according to the technique of the present disclosure.

As described above, in the second embodiment, the screen output control unit 48 displays the character information 22 on the health management screen 100 only in a case where there is the instruction to select the display switching button 175 which is an instruction to display the character information 22. Therefore, in the situation where the owner OW cannot browse the health management screen 100 similar to the precaution information 19 of the first embodiment, it is possible to check the character information 22 by displaying the marks 121 and 123. Conversely, in the situation where the owner OW can browse the health management screen 100, it is possible to perform the non-display of the marks 121 and 123. Therefore, it is possible to effectively keep the character information 22 confidential from the owner OW.

In a case where the character of the pet PT is evaluated as relatively poor, such as the level of gentleness is relatively low, the owner OW who treats the pet PT like a child may seem unpleasant in a case where the character information 22 is known to the owner OW. Therefore, keeping the character information 22 confidential from the owner OW in addition to the precaution information 19 is advantageous for the veterinarian DR and the animal hospital AH.

The bars 120 and 122 and further letters of “gentleness”, “fear degree”, and “character” may be switched between display and non-display in addition to the marks 121 and 123.

Here, the gentleness can be rephrased as tendency to act violently, a degree of handling difficulty, and the like. Similarly, the obedience degree illustrated in FIG. 34 and the like can be rephrased as a rebellion degree. However, it is preferable to use soft image terms such as the gentleness and the obedience degree considering a case where the owner OW browses, rather than intentionally use negative image terms such as the tendency to act violently, the degree of handling difficulty, the rebellion degree.

### Third Embodiment

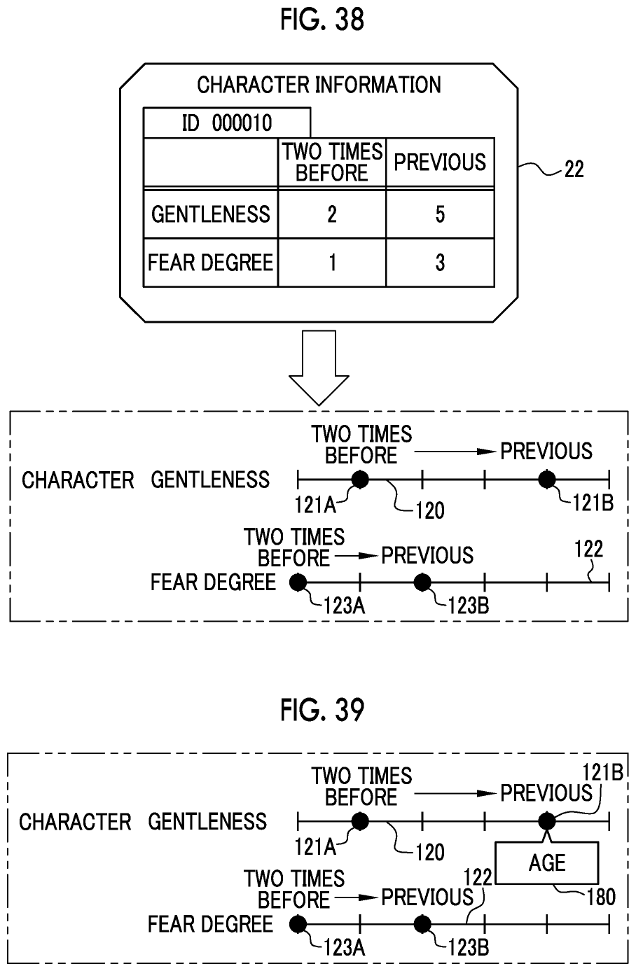

In a third embodiment shown in FIGS. 38 to 40, a transition in the character information 22 is displayed.

In the third embodiment, the veterinarian DR registers the levels of gentleness and fear degree in each time of medical care. The levels of gentleness and fear degree registered in medical care two times before and previous medical care are set as the character information 22.

As shown in FIG. 38, in the third embodiment, a mark 121A indicating the level of gentleness registered in the medical care two times before and a mark 121B indicating the level of gentleness registered in the previous medical care are displayed on the bar 120 indicating the level of gentleness. Similarly, a mark 123A indicating the level of fear degree registered in the medical care two times before and a mark 123B indicating the level of fear degree registered in the previous medical care are displayed on the bar 122 indicating the level of fear degree.

FIG. 38 illustrates a case where the level of gentleness registered in the medical care two times before is 2 and the level of gentleness registered in the previous medical care is 5. FIG. 38 illustrates also a case where the level of fear degree registered in the medical care two times before is 1 and the level of fear degree registered in the previous medical care is 3.

As described above, in the third embodiment, the screen output control unit 48 displays the transition in the character information 22 on the health management screen 100. Therefore, it is possible for the veterinarian DR to grasp the transition in the character of the pet PT. As illustrated in FIG. 38, in a case where the character changes suddenly, such as in a case where the character in the previous time suddenly becomes gentle compared with the character in the previous two times, it is considered that some kind of illness is the cause. Therefore, the transition therein can be a trigger for suspicion of a possible illness and thus can contribute somewhat to early detection of the illness.

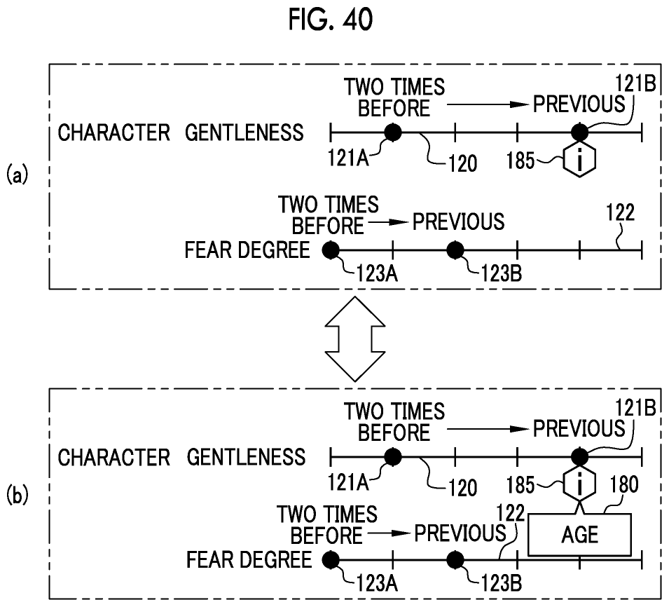

The cause of the transition in the character information 22 determined by the veterinarian DR may be composed to be input through the doctor terminal 11. In this case, it is preferable to display the cause of the transition in the character information 22 determined and input by the veterinarian DR in a balloon display region 180 as shown in FIG. 39. Alternatively, as shown in (a) of FIG. 40, only an information mark 185 is normally displayed and the balloon display region 180 may be displayed as shown in (b) of FIG. 40 only in a case where the information mark 185 is selected, similar to the precaution information 19. FIGS. 39 and 40 illustrate a case where the cause of the transition in the level of gentleness determined and input by the veterinarian DR is “aging”. In a case where the cause of the transition in the character information 22 determined and input by the veterinarian DR is displayed in this way, it is possible to immediately check a reason for determining the cause of a change in character.

The change in character also calms down as the age increases. Therefore, for example, in a case of a pet PT whose age is 6-year-old or more, the display of the transition in the character information 22 may be stopped.

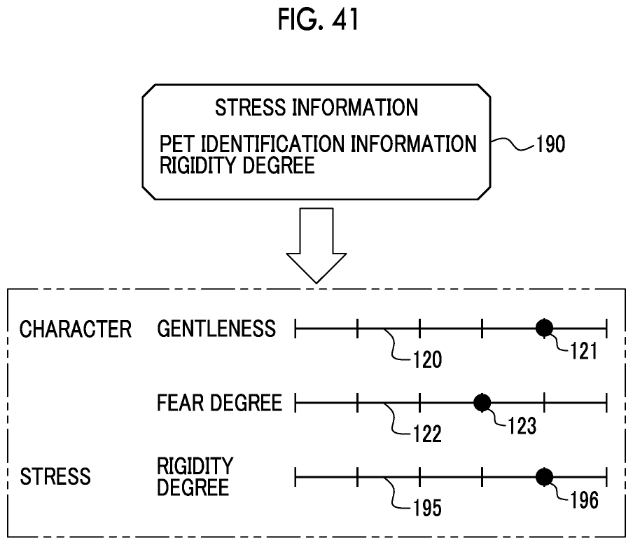

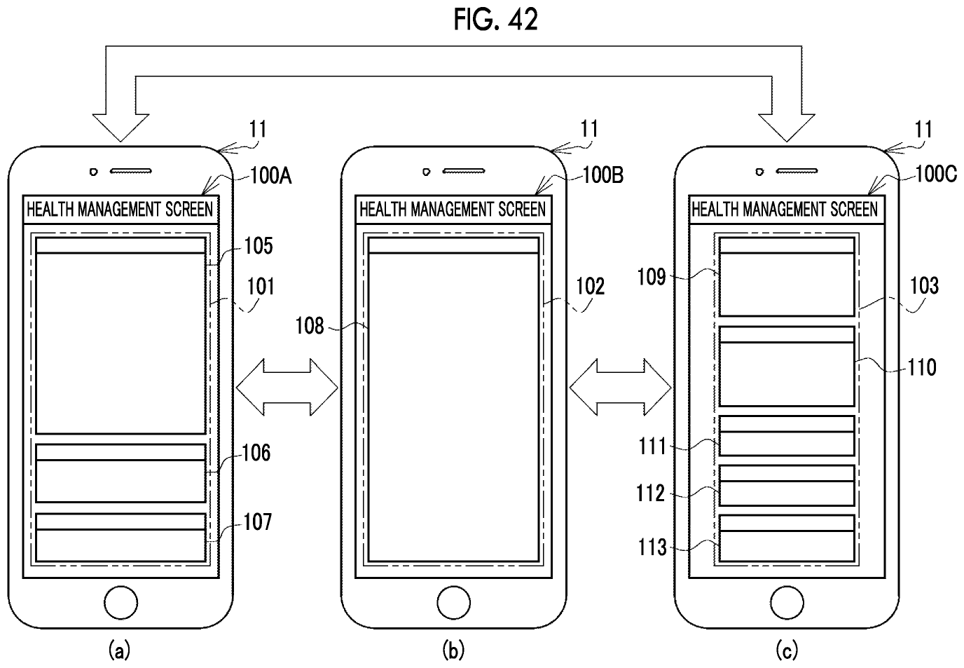

### Fourth Embodiment In a fourth embodiment shown in FIG. 41, stress information 190 of the pet PT is added to the pet information 17.

In FIG. 41, the pet identification information and a rigidity degree are registered in the stress information 190. The rigidity degree is a degree of body stiffness of the pet PT in a case where the pet PT is touched for medical care, expressed by a six-level evaluation of levels one to six, and a more stiff body is at a higher level.

In the fourth embodiment, a bar 195 and a mark 196 indicating the level of rigidity degree of the stress information 190 are displayed below the bar 120 and the mark 121 indicating the level of gentleness of the character information 22 and the bar 122 and the mark 123 indicating the level of fear degree. The bar 195 has “level 1” at the left end and “level 6” at the right end, similar to the bars 120 and 122. FIG. 41 illustrates a case where the rigidity degree is “level 5”.

As described above, in the fourth embodiment, the stress information 190 is added to the pet information 17. Therefore, the medical care can be performed with reference to not only the character of the pet PT but also the stress felt by the pet PT. For example, in a case of a pet PT that is easy to feel the stress, it is possible to take care to reduce the stress as much as possible by calling out.

Instead of or in addition to the rigidity degree, a tremor degree or the like may be registered as a stress item of the stress information 190. The comment may be registered in the stress information 190 in the same manner as the comment is registered in the character information 22 shown in FIGS. 32 and 33. Further, the stress item to be registered in the stress information 190 may be composed to be selectable in the same manner as the character to be registered in the character information 22 is composed to be selectable shown in FIG. 36.

In each embodiment, the desktop personal computer has been exemplified as the doctor terminal 11, but the present invention is not limited thereto. A notebook personal computer, a smartphone, or a tablet terminal may be used as the doctor terminal 11.

In a case where the size of the display 34B is relatively small, such as the smartphone or the tablet terminal, the health management screen 100 shown in FIG. 18 may be small and difficult to view in a case where the screen is displayed as it is. In a case where the doctor terminal 11 is the smartphone, the tablet terminal, or the like, it is preferable to display the health management screen 100 separately for respective sections 101 to 103 as shown in (a), (b), and (c) of FIG. 42.

In (a), (b), and (c) of FIG. 42, in a case where the doctor terminal 11 is the smartphone, the screen output control unit 48 divides the health management screen 100 into three of a health management screen 100A shown in (a) FIG. 42, a health management screen 100B shown in (b) of FIG. 42, and a health management screen 100C shown in (c) of FIG. 42. The health management screen 100A is composed of the first section 101 including the first display region 105 to the third display region 107. The health management screen 100B is composed of the second section 102 including the fourth display region 108. The health management screen 100C includes the third section 103 including the fifth display region 109 to the ninth display region 113. The display of these health management screens 100A to 100C can be switched by, for example, a flick operation as indicated by arrows.

In this manner, in a case where the doctor terminal 11 has a relatively small display 34B such as the smartphone or the tablet terminal, the health management screen 100 is divided and displayed in a switchable manner. Therefore, it is possible to ensure visibility of the health management screen 100.

For example, the name of the pet PT may be commonly displayed on all of the health management screens 100A to 100C. The display contents of the health management screens 100A to 100C may be composed to be settable by the veterinarian DR, such as deleting the fifth display region 109 where the pet food information 24 of the health management screen 100C is displayed.

The medical care information 15 such as the precaution information 19 may be input by a nurse or the like instead of the veterinarian DR based on a memo or the like handwritten by the veterinarian DR.

The animal species of the pet PT is not limited to the dog and the cat exemplified in each embodiment. A bird, a rabbit, a hamster, and the like may be used.

Various modifications are possible for a hardware configuration of the computer constituting the medical care support server 10. For example, the medical care support server 10 may be composed of a plurality of computers separated as hardware for a purpose of improving processing capability and reliability. Specifically, the functions of the reception unit 45, the collation unit 46, and the RW control unit 47, and the function of the screen output control unit 48 are distributed to two server computers. In this case, the medical care support server 10 is composed of two server computers. The doctor terminal 11 may have the function of the medical care support server 10.

As described above, the hardware configuration of the computer can be changed as appropriate according to required performance such as processing capability, safety, and reliability. It is needless to say that not only the hardware but also the AP such as the operation program 40 can be duplicated or distributed and stored in a plurality of storage devices for a purpose of ensuring safety and reliability.

In each embodiment, the medical care support server 10 is used in one animal hospital AH, but the medical care support server 10 may be used in a plurality of animal hospitals AH. In this case, the medical care support server 10 is communicably connected to a plurality of doctor terminals 11 installed in the plurality of animal hospitals AH, for example, through a wide area network (WAN) such as the Internet or a public communication network. Then, the request for distributing the medical care information 15 and the health management screen 100 from the doctor terminal 11 of each animal hospital AH or the like is transmitted to the medical care support server 10 through the WAN, and the medical care information 15 from each animal hospital AH is managed and the health management screen 100 is distributed to the doctor terminal 11 of each animal hospital AH. In this case, an installation place and operation entity of the medical care support server 10 may be a data center operated by a company different from the animal hospital AH, or may be one of the plurality of animal hospitals AH.

In each embodiment, The following various processors can be used as hardware structures of the processing units that execute various pieces of processing, such as the reception unit 45, the collation unit 46, the RW control unit 47, and the screen output control unit 48 (screen generation unit 49 and screen distribution unit 50). The various processors include a programmable logic device (PLD) which is a processor whose circuit configuration is changeable after manufacturing such as a field programmable gate array (FPGA) and/or a dedicated electric circuit which is a processor having a circuit configuration exclusively designed to execute specific processing such as an application specific integrated circuit (ASIC), and the like, in addition to the CPU 32A which is a general-purpose processor that executes software to function as various processing units.

One processing unit may be composed of one of these various processors, or a combination of two or more processors having the same type or different types (for example, a combination of a plurality of FPGAs and/or a combination of a CPU and an FPGA). A plurality of processing units may be composed of one processor.

As an example of composing the plurality of processing units with one processor, first, there is a form in which one processor is composed of a combination of one or more CPUs and software and the processor functions as the plurality of processing units, as represented by a computer such as a client and a server. Second, there is a form in which a processor that realizes the functions of the entire system including the plurality of processing units with one integrated circuit (IC) chip is used, as represented by a system on chip (SoC) or the like. As described above, the various processing units are composed using one or more of the above various processors as the hardware structure.

More specifically, a circuitry in which circuit elements such as semiconductor elements are combined can be used as the hardware structure of these various processors.

From the above description, it is possible to grasp the invention described in the following additional item 1.

Additional Item 1
A medical care support device comprising:
a reception processor that receives precaution information on an owner of a pet as owner information;
a storage control processor that performs control to store the precaution information; and
a screen output control processor that performs control to output a health management screen of the pet, on which a display region dedicated to the precaution information is provided.

It is also possible to combine the various embodiments and/or various modification examples described above as appropriate in the technique of the present disclosure. Further, it is needless to say that the technique thereof is not limited to each embodiment and various configurations may be employed without departing from the gist of the technique thereof. Furthermore, the technique of the present disclosure extends to a storage medium that stores a program non-temporarily in addition to the program.

The described contents and the illustrated contents described above are detailed descriptions of the portions according to the technique of the present disclosure and are merely examples of the technique of the present disclosure. For example, the above descriptions relating to the configuration, function, action, and effect are descriptions relating to examples of the configuration, function, action, and effect of the portions according to the technique of the present disclosure. Accordingly, it is needless to say that an unnecessary portion may be deleted, a new element may be added, or replaced with respect to the described contents and the illustrated contents described above within a range of not departing from the spirit of the technique of the present disclosure. In order to avoid complications and facilitate understanding of the portions according to the technique of the present disclosure, descriptions of common technical knowledge and the like, for which any particular description is not required to enable the implementation of the technique of the present disclosure, are omitted in the described contents and the illustrated contents described above.

In the present specification, “A and/or B” is synonymous with “at least one of A or B”. That is, “A and/or B” means that only A, only B, or a combination of A and B may be used. In the present specification, the same concept as “A and/or B” is adapted also in a case where three or more things are connected and expressed by “and/or”.

All documents, patent applications, and technical standards described in the specification are incorporated by reference in the present specification to the same extent as in a case where individual documents, patent applications, and technical standards are specifically and individually stated to be incorporated by reference.

What is claimed is:

1. A medical care support device comprising:
a reception unit that receives precaution information on an owner of a pet as owner information;
a storage control unit that performs control to store the precaution information; and
a screen output control unit that performs control to output a health management screen of the pet, on which a display region dedicated to the precaution information is provided.

2. The medical care support device according to claim 1,
wherein the screen output control unit displays the display region only in a case where there is an instruction to display the precaution information.

3. The medical care support device according to claim 2,
wherein the screen output control unit displays a mark indicating presence of the precaution information on the health management screen in a state where the display region is not displayed.

4. The medical care support device according to claim 1,
wherein the precaution information is input by being selected from a plurality of options prepared in advance.

5. The medical care support device according to claim 1,
wherein the reception unit receives pet information which is information relating to the pet,
wherein the storage control unit performs control to store the owner information and the pet information in association with each other, and
wherein the screen output control unit displays the owner information and the pet information on the health management screen.

6. The medical care support device according to claim 5,
wherein the pet information includes medical record information of the pet.

7. The medical care support device according to claim 5,
wherein the pet information includes character information of the pet.

8. The medical care support device according to claim 7,
wherein the screen output control unit displays the character information on the health management screen only in a case where there is an instruction to display the character information.

9. The medical care support device according to claim 7,
wherein the screen output control unit displays a transition in the character information on the health management screen.

10. The medical care support device according to claim 5,
wherein the pet information includes breeding environment information of the pet.

11. The medical care support device according to claim 5,
wherein the pet information includes stress information of the pet.

12. The medical care support device according to claim 1,
wherein there is a browsing restriction on the health management screen by authentication, and
wherein the screen output control unit outputs the health management screen only in a case where the authentication is permitted.

13. An operation method of a medical care support device comprising:
a reception step of receiving precaution information on an owner of a pet as owner information;
a storage control step of performing control to store the precaution information; and
a screen output control step of performing control to output a health management screen of the pet, on which a display region dedicated to the precaution information is provided.

14. An operation program of a medical care support device that causes a computer to function as the followings:
a reception unit that receives precaution information on an owner of a pet as owner information;
a storage control unit that performs control to store the precaution information; and
a screen output control unit that performs control to output a health management screen of the pet, on which a display region dedicated to the precaution information is provided.
